(19) United States
(12) Patent Application Publication
(10) Pub. No.: US 2014/0377357 A1
(43) Pub. Date: Dec. 25, 2014

(54) Poloxamer Based Inhalation Composition

(71) Applicant: Daniel Banov, Sugar Land, TX (US)

(72) Inventor: Daniel Banov, Sugar Land, TX (US)

(73) Assignee: Professional Compounding Centers of America, Houston, TX (US)

(21) Appl. No.: 13/921,752

(22) Filed: Jun. 19, 2013

(51) Int. Cl. A61K 47/10; A61K 9/00; A61K 31/7056; A61K 31/5383; A61K 31/573

(52) U.S. Cl. CPC A61K 47/10; A61K 31/5383; A61K 31/573; A61K 31/7056; A61K 9/0073
USPC 424/489; 514/24

(57) ABSTRACT

An inhalation composition for the treatment of bacteria related diseases is provided. The disclosed composition may include a mixture of three or more API(s) and a micronized poloxamer composition. Micronized poloxamer composition may include poloxamer 188 and poloxamer 407. According to an embodiment, an inhalation composition including one or more APIs may be delivered to the respiratory tract by employing inhalation devices, such as inhalers and nebulizers. Antibiotic inhalation composition may provide improved solubility and bioavailability for three or more API(s), such as levofloxacin, betamethasone, and clindamycin. Furthermore, the synergistic effect of micronized poloxamer composition may provide improved solubility and bioavailability of any suitable API.

### CROSS-REFERENCE TO RELATED APPLICATIONS

This application is related to U.S. Utility application Ser. No. 13/921690, entitled Levofloxacin Inhalation Composition, and U.S. Utility application Ser. No. 13/921730, entitled Inhalation Composition for Treating Respiratory Tract Infections, filed on even date herewith.

### BACKGROUND

1. Field of the Disclosure

The present disclosure relates in general to therapeutic formulations, and more particularly, to a poloxamer based inhalation composition.

2. Background Information

Antibiotics are substances used for stopping and treating infections from harmful microorganisms. Antibiotics are used in different forms, such as ointments, creams, gels, pills, sprays, or administered directly into the body by absorption into the bloodstream. The administration method of an antibiotic usually determines how effective the treatment can be, however, it may also determine how severe the side effects may be.

The administration of a drug by inhalation is called a local treatment effected by a direct application of the drug to the affected area and may be expected to produce fewer side effects as compared with the general administration of a drug. However, the application of a drug by inhalation to the respiratory apparatus inclusive of naris, throat, trachea, and lung, may sometimes result in insufficient absorption of the drug through the mucous membrane depending upon the drug. Therefore, inhalation treatments are at a disadvantage in being unable to achieve enough indirect remedial effect attributable to an increase of the concentration of the drug in the blood. Additionally, it may be impractical to administer some drugs by inhalation, as they irritate the mucous membrane, for instance of the respiratory tracts of the bronchi, causing coughing.

For the aforementioned reasons, there is a need for drugs with increased absorption through the mucous membranes of the respiratory apparatus, improved dispersibility to the peripheral airways and alveoli, and which may have reduced side effects.

### SUMMARY

The present disclosure may include a therapeutic formulation for the treatment of bacterial infections in the respiratory tract. The formulation may be employed as an inhalation composition. A method for preparing such composition is also described here. The disclosed inhalation composition may include one or more active pharmaceutical ingredients (APIs) and a combination of two or more poloxamers as excipients. According to an embodiment, a first API may be levofloxacin, a second API may be betamethasone, and a third API may be clindamycin. Poloxamers may include poloxamer 188 and poloxamer 407, among others.

The disclosed inhalation composition may be used for treating bacterial infections caused by microorganisms, such as Bordetella pertussis, Streptococcus pneumoniae, Mycoplasma pneumoniae, Legionella pneumophila, and Chlamydia psittaci, among others. Furthermore, the synergistic effect of micronized poloxamer composition may provide improved solubility and bioavailability of any suitable API, thus decreasing treatment time and side effects occurrence. According to an embodiment, the manufacturing method for inhalation composition may include a non-contact mixing technology. This technology may include an apparatus for applying low-frequency acoustic field, in order to facilitate the mixing process. Furthermore, this approach may allow creating micro-mixing zones through an entire mixing vessel, and therefore, it may allow providing a faster, more uniform mixing throughout a vessel.

Numerous other aspects, features and benefits of the present disclosure may be made apparent from the following detailed description taken together with the drawing figures.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 is a micronized poloxamer composition block diagram, according to an embodiment.

### DETAILED DESCRIPTION

The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

### Definitions

As used here, the following terms may have the following definitions:

“Antibiotic” refers to an agent that destroys or inhibits bacterial growth.

“Excipient” refers to a substance added to a therapeutic formulation in order to provide consistency or form the formulation.

“Poloxamer” refers to a non-ionic triblock copolymer having surfactant properties. Poloxamers may be used as thickening agents, gel formers, co-emulsifiers, solubilizers, and consistency enhancers in creams and liquid emulsions.

“Microprilling” refers to a process where solid spherical microprills may be produced from liquid, tablets or encapsulated ingredients having a diameter of a few microns.

“Minimum inhibitory concentration (MIC)” refers to the lowest concentration of an antimicrobial that will inhibit the visible growth of a microorganism after overnight incubation.

### Description

The present disclosure may relate to a composition of ingredients that, in one embodiment may be an antibiotic composition. The composition may include a combination of two or more poloxamers as excipients and a first API, such as levofloxacin, a second API, such as betamethasone, and a third API, such as clindamycin. According to an embodiment, disclosed composition may be employed as an inhalation formulation for the treatment of bacterial infections in the respiratory tract.

Poloxamer Composition

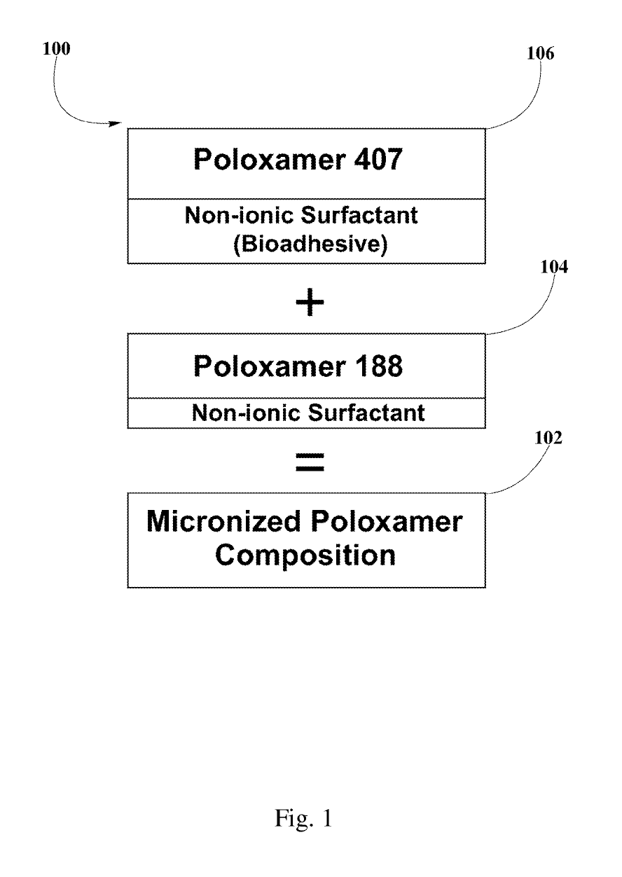

FIG. 1 is a micronized poloxamer composition block diagram 100. The present disclosure may refer to an inhalation composition used for treating bacterial infections in the respiratory tract. The disclosed inhalation composition may include a micronized poloxamer composition 102 as excipient. According to some embodiments, micronized poloxamer composition 102 may include poloxamer 188 104 and poloxamer 407 106, which may be employed for treating bacterial infections. Poloxamer 188 104 may be included in amounts of about 0.1% by weight to about 5%, by weight with about 1% by weight being preferred, and poloxamer 407 106 in amounts of about 0.5% by weight to about 5%, with 1% by weight being preferred.

The benefits of the microprilling process in micronized poloxamer composition 102 may include stronger solubilization properties, controlled dissolution rate, reduction of die-wall friction, achievement of homogeneous blend, elimination of dose dumping, and effectiveness as water soluble lubricant. Micronized poloxamer composition 102 may include surfactant properties, where micronized poloxamer composition 102 may reduce the surface tension or the tension at the interface between any suitable solvent, such as water, and components, such as APIs. Additionally, surfactant agents, such as micronized poloxamer composition 102, may include cleaning properties and may work as surface tension depressants, detergents, dispersing agents, and emulsifiers with in any suitable composition, such as the disclosed inhalation composition.

Furthermore, the disclosed inhalation composition may exhibit solubility properties dictated by the hydrophobic portion of the poloxamers. The use of poloxamers may increase the solubility of the active pharmaceutical ingredient that is employed, thus the drug may have enhanced treatment properties. Furthermore, the properties of each poloxamer may vary in terms of molecular weight, appearance, hydrophilicity/hydrophobicity, and solubility, which may be determined by the chain length of the polyxyethylene (EO-) units and polyoxypropyene (PO-) units.

According to an embodiment, micronized poloxamer composition 102 in combination with a suitable API, may decrease the minimum inhibitory concentration (MIC) for microorganisms such as Escherichia coli, Staphylococcus aureus, Pseudomonas aeruginosa, Candida albicans, Aspergillus niger, Salmonella typhimurium, methicillin resistant Staphylococcus aureus, Aspergillus fumigatus, and Rhizopus oryzae, among others. This may be achieved by allowing a more uniform dispersion as a result of the narrow distribution of particles from an API.

Manufacturing Method for Micronized Poloxamer Composition

According to an embodiment, various components are combined to form micronized poloxamer composition 102.

The manufacturing method for micronized poloxamer composition 102 may include a non-contact mixing technology. This technology may include an apparatus for applying low-frequency acoustic field, in order to facilitate the mixing process. Furthermore, this approach may allow creating micro-mixing zones through an entire mixing vessel, and therefore, it may allow providing a faster, more uniform mixing throughout a vessel.

According to an embodiment, micronized poloxamer composition 102 may be obtained in powder form having a particle size between about 30 μm and about 70 μm, where 50 μm may be preferred. The powder may be employed to fill capsules, which may be used for inhalation of the composition.

According to an embodiment, inhalation composition in solution form may include from about 2 ml to about 10 ml of a solvent, while an antibiotic compound may include a levofloxacin dose ranging from about 50 mg to about 150 mg, a betamethasone dose ranging from about 0.1 mg to about 0.5 mg, and a clindamycin dose ranging from about 50 mg to about 300 mg.

The disclosed inhalation composition may be delivered to the respiratory tract employing devices, such as metered-dose inhalers (MDIs), dry powder inhalers, aerosols, and nebulizers, among others. By administering the disclosed composition via inhalation, the drug may be driven directly into the respiratory tract and less may be absorbed into the bloodstream, therefore increasing bioavailability of the medication and decreasing treatment time. Furthermore, as inhalation composition may have a small particle size, the solubility of the medicine may be improved, hence enhancing the action of the inhalation composition. Additionally, inhalation composition may reduce API's side effects, such as dizziness, fainting, sudden pain, confusion, insomnia, and severe headache, among others.

APIs

An antibiotic agent may be employed as an API. According to an embodiment, micronized poloxamer composition 102 may be combined with one or more APIs to produce antibiotic inhalation composition. Antibiotic inhalation composition may be efficient and effective in treating lung related bacterial infections caused by bacteria, such as Bordetella pertussis, Streptococcus pneumoniae, Mycoplasma pneumoniae, Legionella pneumophila, and Chlamydia psittaci, among others.

Levofloxacin

Levofloxacin is an antibiotic of the fluoroquinolone drug class. The spectrum of activity for this drug includes several bacterial pathogens (e.g. Escherichia coli, Haemophilus influenzae, Klebsiella pneumoniae, Legionella pneumophila, Moraxella catarrhalis, Proteus mirabilis, Pseudomonas aeruginosa, Staphylococcus aureus, Streptococcus pneumoniae, Staphylococcus epidermidis, Enterococcus faecalis, and Streptococcus pyogenes).

Levofloxacin may be used to treat infections, such as pneumonia, chronic bronchitis and sinues, urinary tract, kidney, prostate, and skin infections. Levofloxacin may also be used to treat people who have been exposed to anthrax germs. Furthermore, levofloxacin may also be used to treat endocarditis, sexually transmitted diseases, and tuberculosis (TB). Levofloxacin is also used to prevent or treat traveler's diarrhea and plague.

Betamethasone

Betamethasone is a corticosteroid used for treating tissue irritation, such as itching and flaking from eczema in skin and inflammation in the respiratory system. Corticosteroids are generally used to prevent the progression of inflammation in vital organs, which may result in an organ failure and subsequently, to death. Furthermore, corticosteroids, such as betamethasone may be used to relief patients with rheumatoid arthritis from pain and stiffness.

Inhaled betamethasone may be used as a first-line therapy for reducing airway inflammation and may include advantages over oral preparations. Inhalation allows a direct route of delivery to the respiratory tract.

Clindamycin

Clindamycin is an antibiotic of the lincosamides drug class. Clindamycin compositions are usually used for preventing bacteria from replicating, by interfering with the synthesis of proteins. The spectrum of activity for this drug includes aerobic and anaerobic organisms. Some aerobic organisms may include Staphylococcus and Streptococcus, while anaerobic organisms may include fusobacterium, bacteroides, and prevotella.

Clindamycin may be inhaled for treating anaerobic infections in the respiratory tract. Furthermore, may be used to treat infections caused by aerobic bacteria. It may be used to treat joint or bone infections caused by organisms, such as Staphylococcus aureous. The usage of clindamycin for treating acne is also common

### EXAMPLES

Example #1 is an embodiment of antibiotic inhalation composition, where instead of employing poloxamer 188 104 and poloxamer 407 106 in micronized poloxamer composition 102, other poloxamers may be used. Poloxamers may include: poloxamer 101, poloxamer 105, poloxamer 108, poloxamer 122, poloxamer 124, poloxamer 181, poloxamer 182, poloxamer 183, poloxamer 184, poloxamer 185, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and combinations thereof.

Example #2 is an embodiment of micronized poloxamer composition 102, where micronized poloxamer composition 102 may be used in combination with xylitol or sugar alcohol. Xylitol may be included in amounts of about 50% by weight to about 90% by weight, most suitable being 80% by weight.

While various aspects and embodiments have been disclosed here, other aspects and embodiments may be contemplated. The various aspects and embodiments disclosed here are for purposes of illustration and are not intended to be limiting, with the true scope and spirit being indicated by the following claims.

What is claimed is:

1. A composition for prevention and treatment of infections of the respiratory tract caused by bacteria, comprising levofloxacin, betamethasone, clindamycin and at least two poloxamers.

2. The composition according to claim 1, wherein one of the at least two poloxamers is selected from the group consisting of poloxamer 188, poloxamer 407, and combinations thereof.

3. The composition according to claim 1, wherein one of the at least two poloxamers is poloxamer 188.

4. The composition according to claim 3, wherein the poloxamer 188 is about 0.1% by weight to about 5% by weight.

5. The composition according to claim 3, wherein the poloxamer 188 is about 1% by weight.

6. The composition according to claim 1, wherein one of the at least two poloxamers is poloxamer 407.

7. The composition according to claim 6, wherein the poloxamer 407 is about 0.1% by weight to about 5% by weight.

8. The composition according to claim 6, wherein the poloxamer 407 is about 1% by weight.

9. The composition according to claim 1, wherein one of the at least two poloxamers is micronized.

10. The composition according to claim 1, wherein one of the at least two poloxamers comprises a particle size of about 30 μm to about 70 μm.

11. The composition according to claim 1, wherein one of the at least two poloxamers comprises a particle size of about 50 μm.

12. A method for prevention and treatment of infections of the respiratory tract caused by bacteria, comprising administering to a patient in need of such treatment a formulation comprising levofloxacin, betamethasone, clindamycin, and at least two poloxamers.

13. The method according to claim 12, wherein one of the at least two poloxamers is selected from the group consisting of poloxamer 188, poloxamer 407, and combinations thereof.

14. The method according to claim 12, wherein one of the at least two poloxamers is poloxamer 188.

15. The method according to claim 14, wherein the poloxamer 188 is about 0.1% by weight to about 5% by weight.

16. The method according to claim 14, wherein the poloxamer 188 is about 1% by weight.

17. The method according to claim 12, wherein one of the at least two poloxamers is poloxamer 407.

18. The method according to claim 17, wherein the poloxamer 407 is about 0.1% by weight to about 5% by weight.

19. The method according to claim 17, wherein the poloxamer 407 is about 1% by weight.

20. The method according to claim 12, wherein the formulation is a powder.

21. The method according to claim 20, wherein the powder is dissolved in a solvent comprising saline.

22. The method according to claim 12, wherein the formulation is administered using an inhalation device selected from the group consisting of a metered-dose inhalers (MDIs), a dry powder inhalers, and a nebulizer.

22. The method according to claim 12, wherein the formulation is administered using one selected from the group consisting of a syringe, pipette, measured spoon, and eyedropper.

23. The method according to claim 12, wherein the infections of the respiratory tract are selected from the group consisting of Bordetella pertussis, Streptococcus pneumoniae, Mycoplasma pneumoniae, Legionella pneumophila, and Chlamydia psittaci.
